(19) United States
(12) Patent Application Publication
(10) Pub. No.: US 2005/0034733 A1
(43) Pub. Date: Feb. 17, 2005

(54) System and method for fabricating an interim dental guard device

(75) Inventors: Katherine Evenson Liddle, Cape May Court House, NJ (US); Wolfgang Muehlbauer, Hamburg (DE)

Correspondence Address: KENYON & KENYON, ONE BROADWAY, NEW YORK, NY, 10004, US

(73) Assignee: ALL DENTAL PRODX, LLC

(21) Appl. No.: 10/640,782

(22) Filed: Aug. 13, 2003

(51) Int. Cl. A61F 5/56; A61C 5/14

(52) U.S. Cl. 128/859; 128/848; 128/861

(57) ABSTRACT

A system and method for fabricating an interim dental guard device for preventing or reducing damage caused by bruxism to existing dentition and/or restorative dental work. The device is composed of a material that, when heated, is moldable around at least a portion of a patient's dental arch so as to form an impression of at least a portion of the dental arch, and that is solid at about normal body temperature so as to retain the impression once formed.

### FIELD OF THE INVENTION

The present invention relates to an interim dental guard device. More specifically, the present invention relates to a system and method for fabricating an interim dental guard device for preventing or reducing damage caused by bruxism.

### BACKGROUND INFORMATION

Bruxism is a medical condition characterized by a person unconsciously, e.g., during sleep, grinding his or her teeth or clenching his or her jaw. While preventing bruxism is an important consideration for a person during any period of time, it is of particular importance when a person has undergone a restorative dental procedure.

In order to prevent bruxism, a dental guard device may be worn. Such dental guard devices are well known and typically are referred to as “night guards” (it is noted that, despite being referred to as “night guards,” these devices need not be worn exclusively at night). In order to fabricate a conventional night guard, an impression of a patient's mouth is made at a dental office, and the impression is sent to a laboratory. The laboratory forms an initial version of the night guard of a suitably durable material. The initial night guard is sent back to the dental office and the dentist checks the fit of the same in the patient's mouth. Several appointments may be necessary in order to ensure that a proper fit is achieved. Once the fit of the night guard is finalized, the permanent night guard may be worn indefinitely.

Conventional night guards are time-consuming to fabricate. However, there are situations in which protection against bruxism is immediately desired. For instance, a restorative dental procedure may involve numerous appointments and take several weeks or months to perform. If a patient grinds his or her teeth or clenches his or her jaw during this time period, the restorative dental procedure may be ruined. Furthermore, after each of these appointments, the patient's restorative dental work and/or existing dentition may be shaped differently, and thus it would be wasteful to fabricate a night guard in the conventional manner only to have the night guard fit improperly after a patient undergoes subsequent treatment. Further still, when the restorative dental procedure is completed, a significant amount of time may be required after taking an impression of the patient's mouth before the permanent night guard is fabricated by the laboratory and adjusted to fit properly.

Thus, there is a need for a system and method for efficiently fabricating an interim dental guard device for preventing or reducing the damage caused by bruxism.

### SUMMARY OF THE INVENTION

The present invention, according to one example embodiment thereof, relates to a system and method for fabricating an interim dental guard device for preventing or reducing damage caused by bruxism to existing dentition and/or restorative dental work. The device is composed of a material that, when heated, is moldable around at least a portion of a patient's dental arch so as to form an impression thereof and that is solid at about normal body temperature so as to retain the impression once formed.

According to one example method, the present invention is related to a method for fabricating the interim dental guard device for preventing or reducing damage caused by bruxism. The method includes the steps of heating the device in a pre-formed condition to a first temperature in order to soften the same. The softened device is placed into a patient's mouth. The device is then molded around at least a portion of a dental arch of the patient so as to form an impression thereof. The device is cooled to a second temperature at which the impression is retained by the device. In one embodiment, the impression of one arch, or a part of one arch, of the patient may be formed without registering the counterbite of the patient so that an opposite side of the thermoplastic material remains smooth. Alternatively, after being molded to form an impression of a single arch of the patient's existing dentition and/or restorative dental work, the device may be smoothed on the opposite side of the thermoplastic material so as to remove any unintended or undesired impressions left therein by the other arch of the patient.

### BRIEF DESCRIPTION OF THE DRAWINGS

FIG. 1 is a diagram that illustrates an interim dental guard device in a pre-formed condition, in accordance with one embodiment of the present invention; and

FIG. 2 is a diagram that illustrates the interim dental guard device of FIG. 1 in a formed condition.

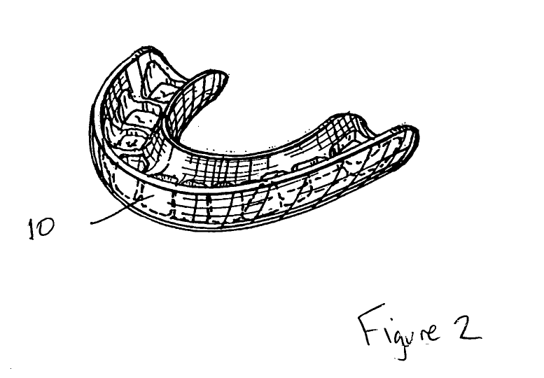

### DETAILED DESCRIPTION

The present invention, in accordance with one embodiment thereof, provides a system and method for fabricating an interim dental guard device for preventing or reducing damage caused by bruxism that can be fabricated for use by a patient in a relatively short amount of time and that is relatively inexpensive to fabricate. For instance, the present invention, provides an interim dental guard device that can be quickly made for use by a patient that is undergoing or that has undergone a restorative dental procedure, so as to prevent the patient from suffering damage caused by bruxism while waiting for a permanent night guard device to be fabricated.

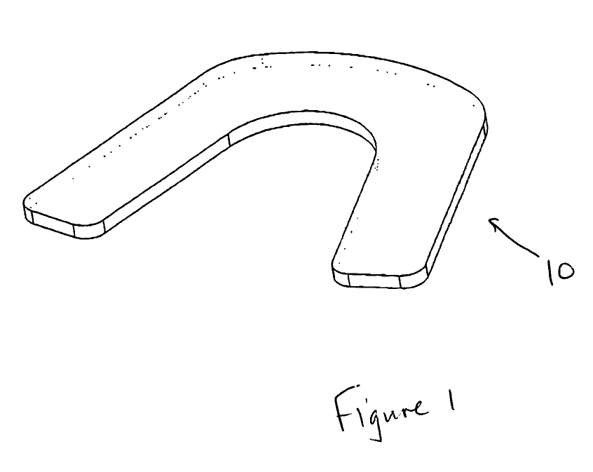

FIG. 1 is a diagram that illustrates an interim dental guard device in accordance with one embodiment of the present invention. Specifically, FIG. 1 illustrates an interim dental guard device 10 in a pre-formed condition. The interim dental guard device 10 may be sized and shaped so as to conform generally to at least a portion of, e.g., a quadrant, or a complete, arch of a patient, e.g., a patient's lower arch of teeth, or more usually a patient's upper arch of teeth. For example, the dental guard device may be curved or U-shaped. Alternatively, the interim dental guard device 10 may be rectangular or some other shape. In order to insure sufficient stability of the interim dental guard device 10, the device desirably has a thickness of about 3.175 mm (⅛″) or more. However, any thickness may be possible provided that it is sufficient to prevent biting through. While FIG. 1 illustrates the interim dental guard device 10 being formed of a single, curved piece, other embodiments are possible in which the interim dental guard device 10 is formed from more than one piece, each piece having one of any conceivable number of shapes. Having the interim dental guard device 10 conform to a significant portion of or a complete arch of the patient provides an advantage in that, due to the size of the device, it is less likely to be inadvertently swallowed by the patient. In addition, having the interim dental guard device 10 conform to a significant portion of or a complete arch of the patient provides the advantage that it may be easier for a user to position and handle the interim dental guard device 10, thereby enabling a better impression of the arch to be obtained, as more fully set forth below.

The interim dental guard device 10 is preferably comprised of a thermoplastic material that is rigid at room temperature but is moldable (e.g., soft enough to have an impression made) when heated. Advantageously, the thermoplastic material of which the interim dental guard device 10 is composed is moldable when heated to a temperature above body temperature, preferably between about 45° C. and about 75° C., more preferably between about 50° C. and about 70° C., and most preferably between about 60° C. and about 65° C. At these temperatures, the material is preferably still sufficiently stable so as to permit it to be handled by a user. In addition, the thermoplastic material of which the interim dental guard device 10 is composed is stable enough to retain a given shape when the thermoplastic material is at a temperature at or below about 37° C., e.g., normal body temperature. In this respect, the thermoplastic material of which the interim dental guard device 10 is composed is moldable when at a temperature which is still tolerable to a patient's mouth, but which is solid enough to retain a given shape, e.g., an impression, when the material is at about normal body temperature.

According to one embodiment of the present invention, the interim dental guard device is comprised of polycaprolactone or polyvinyl acetate, although any type of material may be employed so long as it is configured to receive and retain an impression of a patient's mouth as described hereinbelow. Other examples of suitable materials can be found in U.S. Pat. No. 5,066,231 and Japanese Patent Number 63270759, which are incorporated herein by reference as fully as if set forth in their entirety.

The thermoplastic material of which the interim dental guard device 10 is composed may also include additives. For instance, the thermoplastic material may include additives to change the condition of the material when it is moldable, e.g., at its softening point, such as by providing a more noticeable change in opacity of the material or a change in color of the material when heated to the softening point. The thermoplastic material may also include a color agent, an agent for taste, hydrophilic agents, smoothing agents, agents for improving the adherence of gels or foams to the material, etc.

In use, the interim dental guard device, such as the interim dental guard device 10 illustrated in FIG. 1, is heated to a temperature at which it is moldable. The interim dental guard device 10 is then placed in the mouth of a patient and is molded so as to conform at least partially, and preferably completely, to the existing dentition and/or restorative dental work of the patient. In this manner, an impression of the existing dentition and/or restorative dental work of the patient is formed. According to one embodiment of the present invention, the interim dental guard device 10 is molded so as to conform at least partially, and preferably completely, to the existing dentition and/or restorative dental work of a single arch of the patient. In this embodiment, the impression of the single arch of the patient may be formed with, but preferably without, registering the counterbite of the patient. In other words, in one such preferred embodiment, when the impression of the single arch of the patient is formed on one side of the thermoplastic material, an impression of the other arch of the patient is not formed on the opposite side of the thermoplastic material. Alternatively, after being molded to form an impression of a single arch of the patient's existing dentition and/or restorative dental work, the interim dental guard device 10 may be smoothed on the opposite side of the thermoplastic material so as to remove any unintended or undesired impressions left therein by the other arch of the patient. By refraining from registering the counterbite of the patient, or else by smoothing the opposite side of the interim dental guard device 10 so as to remove any unintended or undesired impressions left therein by the other arch of the patient, the interim dental guard device 10 permits free movement of the patient's jaw. Thus, if a patient experiences bruxism while wearing an interim dental guard device 10 formed in one of these manners, the lower jaw of the patient may move freely with respect to the upper jaw by sliding along the smooth surface on the side of the interim dental guard device 10 that does not have an impression made therein. The damage caused by bruxism to the existing dentition or the restorative dental work of the patient is thereby prevented or at least reduced.

Since the interim dental guard device 10 of the present invention can be fabricated relatively quickly and easily, it can be made in a dental office. Thus, for instance, when a restorative dental procedure involving numerous appointments and/or taking several weeks or months to perform is performed, one or more interim dental guard devices 10 may be quickly and inexpensively formed, e.g., after each appointment, if necessary. If a patient grinds his or her teeth or clenches his or her jaw during this time period, the interim dental guard device 10 may be used to prevent the restorative dental procedure from being ruined. Furthermore, even if after each of these appointments the patient's restorative dental work and/or existing dentition is shaped differently, multiple interim dental guard devices 10 may be employed during the course of the work without incurring excessive costs and without requiring much time or effort to make. Still further, when the restorative dental procedure is completed, an interim dental guard device 10 may be quickly and inexpensively formed and worn by a patient while waiting for a permanent night guard to be fabricated and fitted. Thus, the patient's restorative dental work is prevented from being harmed by bruxism during the time between the dentist taking an impression of the patient's mouth for a permanent night guard and the permanent night guard being fabricated by the laboratory and adjusted to fit properly.

Thus, the several aforementioned objects and advantages of the present invention are most effectively attained. Those skilled in the art will appreciate that numerous modifications of the exemplary example embodiments described hereinabove may be made without departing from the spirit and scope of the invention. Although various exemplary example embodiments of the present invention have been described and disclosed in detail herein, it should be understood that this invention is in no sense limited thereby and that its scope is to be determined by that of the appended claims.

1. An interim dental guard device for preventing or reducing damage caused by bruxism, the device having a first side and a second side and being is composed of a material that is moldable around at least a portion of a patient's dental arch at a first temperature so as to form on the first side an impression of the at least a portion of the dental arch and that is solid at about normal body temperature so as to retain the impression once formed, the second side being smooth.

2. The device of claim 1, wherein the material includes a thermoplastic.

3. The device of claim 2, wherein the thermoplastic is at least one of polycaprolactone and polyvinyl acetate.

4. The device of claim 1, wherein the device has a shape that conforms generally to a shape of the at least a portion of the patient's dental arch.

5. The device of claim 1, wherein the device has a shape that conforms generally to a quadrant of the patient's dental arch.

6. The device of claim 1, wherein the device has a shape that conforms generally to a complete dental arch of the patient.

7. The device of claim 1, wherein the material of which the device is composed is moldable at a temperature between about 45° C. and 75° C.

8. The device of claim 1, wherein the material of which the device is composed is moldable at a temperature between about 50° C. and 70° C.

9. The device of claim 1, wherein the material of which the device is composed is configured to retain a given shape at a temperature of about 37° C.

10. The device of claim 1, wherein the material of which the device is composed includes an additive.

11. The device of claim 10, wherein the additive includes at least one of an additive to change the condition of the material when it is moldable, an additive to provide a more noticeable change in opacity of the material or a change in color of the material when heated to a molding temperature, a color agent, an agent for taste, a hydrophilic agent, a smoothing agent, and an agent for improving the adherence of gels or foams to the material.

12. A method for fabricating an interim dental guard device for preventing or reducing damage caused by bruxism, the device having first and second sides, the method comprising the steps of:
heating the device in a pre-formed condition to a first temperature;
placing the device into a patient's mouth;
molding the device around at least a portion of a dental arch of the patient at the first temperature so as to form on the first side of the device an impression of the at least a portion of the dental arch;
cooling the device to a second temperature at which the impression is retained, wherein the second side of the device is smooth when the device is cool.

13. The method of claim 12, wherein the molding step includes preventing the second side of the device from becoming unsmooth during molding.

14. The method of claim 12, further comprising the step of smoothing the second side of the device during molding.

15. The method of claim 12, further comprising the step of providing the device as a thermoplastic material.

16. The method of claim 12, wherein the step of providing the device as a thermoplastic material includes providing the device as at least one of polycaprolactone and polyvinyl acetate.

17. The method of claim 12, further comprising the step of providing the device with a shape that conforms generally to a shape of the at least a portion of the patient's dental arch.

18. The method of claim 12, further comprising the step of providing the device with a shape that conforms generally to a quadrant of the patient's dental arch.

19. The method of claim 12, further comprising the step of providing the device with a shape that conforms generally to a complete dental arch of the patient.

20. The method of claim 15, wherein the molding step includes molding the device at a temperature between about 45° C. and 75° C.

21. The method of claim 15, wherein the molding step includes molding the device at a temperature between about 50° C. and 70° C.

22. The method of claim 15, wherein the step of cooling the device to a second temperature includes cooling the device to a temperature at or below about 37° C.
